(19) United States
(12) Patent Application Publication
(10) Pub. No.: US 2025/0228444 A1
(43) Pub. Date: Jul. 17, 2025

(54) REMOVABLE TIP ENDOSCOPE

(71) Applicant: EndoChoice, Inc., Alpharetta, GA (US)

(72) Inventors: Avi LEVY, Herzliya (IL); Moshe LEVI, Ganey Tikva (IL); Amram AIZENFELD, Ramot Menashe (IL)

(73) Assignee: EndoChoice, Inc., Alpharetta, GA (US)

(21) Appl. No.: 19/172,179

(22) Filed: Apr. 7, 2025

Related U.S. Application Data
(63) Continuation of application No. 17/685,951, filed on Mar. 3, 2022, now Pat. No. 12,290,241.
Application No. 17/685,951 is a continuation of application No. 16/567,623, filed on Sep. 11, 2019, now Pat. No. 11,291,357.
Application No. 16/567,623 is a continuation of application No. 15/491,811, filed on Apr. 19, 2017, now Pat. No. 10,470,649.
Application No. 15/491,811 is a continuation of application No. 13/713,449, filed on Dec. 13, 2012, now Pat. No. 9,655,502.
(60) Provisional application No. 61/569,796, filed on Dec. 13, 2011.

(51) Int. Cl. A61B 1/05; A61B 1/00; A61B 1/018; A61B 1/06; A61B 1/12

(52) U.S. Cl. CPC A61B 1/053; A61B 1/00045; A61B 1/00105; A61B 1/00114; A61B 1/00181; A61B 1/018; A61B 1/0676; A61B 1/00091; A61B 1/00124; A61B 1/0638; A61B 1/126

(57) ABSTRACT

There is provided herein, an endoscope comprising an elongated shaft terminating with a tip section wherein said tip section comprises a permanent section connected to the elongated shaft and a removable section securely connectable to the permanent section, wherein the removable section comprises at least one capture device and at least one light source.

### CROSS-REFERENCE TO RELATED APPLICATIONS

This application claims the benefit of U.S. Provisional Application No. 61/569,796, filed Dec. 13, 2011, the content of which is expressly incorporated herein by reference in its entirety.

### FIELD OF THE INVENTION

Embodiments of the disclosure relate to a multi camera endoscope with a removable tip section.

### BACKGROUND OF THE INVENTION

Endoscopes have attained great acceptance within the medical community, since they provide a means for performing procedures with minimal patient trauma, while enabling the physician to view the internal anatomy of the patient. Over the years, numerous endoscopes have been developed and categorized according to specific applications, such as cystoscopy, colonoscopy, laparoscopy, upper GI endoscopy and others. Endoscopes may be inserted into the body's natural orifices or through an incision in the skin.

An endoscope is usually an elongated tubular shaft, rigid or flexible, having one or more video cameras or fiber optic lens assemblies at its distal end, or one or more video cameras or fiber optic lens assemblies positioned further back, e.g., more proximally on or near the lateral surface area of the tip section, and point sideways. The shaft is connected to a handle, which sometimes includes an ocular for direct viewing. Viewing is also usually possible via an external screen. Various surgical tools may be inserted through a working channel in the endoscope for performing different surgical procedures.

Among the disadvantages of existing endoscopes, are their limited field of view and their complicated packing of all the required elements, such as electronics and together with fluid carrying elements in the small sized endoscope tip section. In addition, different procedures (for example for different conditions or subjects) may require different endoscope structures and functions.

There is thus a need in the art for endoscopes, such as colonoscopies, that allow a broader field of view and also enable the function of all necessary elements in the tip section.

The foregoing examples of the related art and limitations related therewith are intended to be illustrative and not exclusive. Other limitations of the related art will become apparent to those of skill in the art upon a reading of the specification and a study of the figures.

### SUMMARY OF THE INVENTION

One aspect of the disclosure relates to an endoscope comprising an elongated shaft terminating with a tip section, wherein said tip section comprises: a permanent section connected to the elongated shaft; and a removable section securely connectable to the permanent section, wherein the removable section comprises one or more capture devices and one or more light sources. In some embodiments of the endoscope, the capture device within the removable section comprises a front-facing camera. In some embodiments of the endoscope, the capture device within the removable section comprises a side-facing camera. In some embodiments of the endoscope, the capture device within the removable section comprises a front-facing camera and a side-facing camera. In some embodiments of the endoscope, the permanent section comprises one or more capture devices. In some embodiments, the capture device within the permanent section comprises a side-facing camera. In some embodiments of the endoscope, the tip section comprises a distal face and the permanent section forms a part of the distal face, and the removable section forms another part of the distal face. In some embodiments of the endoscope, the tip section comprises a distal face, and the distal face is fully comprised in the removable section. In some embodiments of the endoscope, the permanent section comprises a hollow elongated section protruding from the permanent section and the removable section comprises a channel configured to continue said hollow elongated section, so as to provide a working channel configured for insertion of a surgical tool. In some embodiments of the endoscope, the removable section comprises a hollow elongated section protruding from the removable section and the permanent section comprises a channel configured to continue said hollow elongated section, so as to provide a working channel configured for insertion of a surgical tool. In some embodiments of the endoscope, the removable section comprises an elongated section configured to pass a cable for electric or data signals, the elongated section ending with a first connector, and the permanent section comprises a recess configured to pass electric and/or data signals, said recess comprising a second connector connectable to the first connector. In some embodiments of the endoscope, the removable section comprises a first optic fiber and a first lens, and the permanent section comprises a second optic fiber and a second lens, such that the first lens corresponds to the second lens so as to transfer light between the first optic fiber and the second optic fiber. In some embodiments of the endoscope, the permanent section comprises an elongated section configured to pass a cable for electric or data signals, the elongated section ending with a first connector, and the removable section comprises a recess configured to pass electric or data signals, the recess comprising a second connector connectable to the first connector. In some embodiments of the endoscope, the endoscope further comprises a switch on the permanent section such that the endoscope can operate only if the switch is pressed by the removable section. In some embodiments of the endoscope, an indication of a status of the switch is displayed on a display device.

Another aspect of the disclosure relates to a removable section of an endoscope tip, the removable section comprising one or more capture devices and one or more light sources, wherein the removable section is connectable to a permanent section of the endoscope tip. In some embodiments of the removable section, the capture device within the removable section comprises a front-facing capture device. In some embodiments of the removable section, the capture device within the removable section comprises a side-facing camera. In some embodiments of the removable section, the capture device within the removable section comprises a front-facing camera and a side-facing camera. In some embodiments, the removable section is configured to form a part of a distal face of the endoscope tip. In some embodiments, the removable section is configured to form a full distal face of the endoscope tip. In some embodiments, the removable section comprises a channel into which a hollow elongated section protruding from the permanent section is configured to be inserted, so as to provide a working channel configured for insertion of a surgical tool. In some embodiments the removable section comprises a hollow elongated section protruding from the removable section, the hollow elongated section is configured to be inserted into a channel in the permanent section, so as to provide a working channel configured for insertion of a surgical tool. In some embodiments, the removable section comprises an elongated section configured to pass a cable for electric or data signals, the elongated section ending with a first connector, connectable to a second connector comprised in a recess in the permanent section. In some embodiments, the removable section comprises a recess configured to pass a cable for electric or data signals, the recess comprising a connector, said connector connectable to a second connector located in an elongated section protruding from the permanent section. In some embodiments, the removable section is adapted to press a switch on the permanent section such that the endoscope can operate only if the switch is pressed by the removable section. In some embodiments the removable section is configured for a single use.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. The figures are listed below.

FIG. 1 shows a semi-pictorial view of an endoscopy system;

FIG. 2 shows a perspective view of a multi-camera endoscope;

FIG. 3 shows a perspective view of a full cross section removable tip section removed from the permanent section, in accordance with some exemplary embodiments of the disclosure;

FIG. 4 shows a perspective view of a full cross section removable tip section attached to the permanent section, in accordance with some exemplary embodiments of the disclosure;

FIG. 5 shows a perspective view of a partial cross section removable tip section removed from the permanent section, in accordance with some exemplary embodiments of the disclosure; and

FIG. 6 shows a perspective view of a partial cross section removable tip section attached to the permanent section, in accordance with some exemplary embodiments of the disclosure.

### DETAILED DESCRIPTION

An aspect of some embodiments relates to an endoscope having a removable multi camera tip section.

The endoscope may comprise a front-pointing camera, fiber optic lens assembly, or another capturing device for capturing the area of interest, and optionally one or more light sources such as a Light Emitting Diode (LED), or one or more fluid injectors, positioned at a distal end of the endoscope for enabling the capturing device to capture images of the body cavity into which the endoscope is inserted. The endoscope may further comprise an opening at its distal end, the opening connected to a working channel, through which an operator may insert a surgical tool in order to remove, treat and/or extract a sample of the findings in the body cavity. In some embodiments, the endoscope may comprise one or more side-pointing capturing devices, and optionally additional light sources or fluid injectors, positioned further back, e.g., more proximally on the endoscope, on or near the lateral surface area of the tip section.

An endoscope is a general term for a wide variety of devices, which are adapted to be used in a variety of applications, such as cystoscopy, colonoscopy, laparoscopy, upper GI endoscopy or others.

According to some embodiments, one technical problem addressed by the disclosed apparatus and method relates to multiple endoscope configurations being required for handling the multiplicity of applications. Different configurations may require different type, number, positioning, directing, focusing or other tuning of the capturing devices, light sources or other components on the endoscope. Therefore, although multiple parts of an endoscope system may be common to many of the configurations, yet multiple endoscopes may be required. This poses significant requirements on a health institute, including for example financial requirements, storage, maintenance, training or the like.

Some different configurations may also be required for different patients or patient types, such as adults, children, babies, or the like.

Some different configurations may also be required for different procedures, such as Colonoscopy, Gastroscopy, Endoscopic Ultrasound (EUS), ERCP or the like.

Yet another technical problem addressed by embodiments of the disclosure relates to maintenance costs. When replacing the camera head, for example due to defective objective lens, the entire colonoscope has to be disassembled, which is an expensive process.

According to some embodiments, a technical solution may be the provisioning of an endoscope having a removable tip section. The tip section may also be partially removable, for example, with a permanent section and a removable section. The removable section of the tip may be removably connected or attached to the permanent section of the tip which is connected to the shaft, so that endoscopes having different configurations can be used with the same system. According to the endoscopic task to be performed, a removable section having an appropriate configuration is selected and connected to the shaft or to the permanent section. When the endoscopy session is over, the removable section of the tip may be removed and another removable section having the same or a different configuration can be connected to the permanent section or to the shaft.

In some embodiments, the removable section of the tip comprises a substantially full cross section of the tip, for example the whole distal surface of the tip, possibly excluding some openings or small parts such as rings. In some of these embodiments, all channels and flows going through the tip, such as optic fibers, power supply, water supply, data lines transferring images, working channels for transferring equipment, or the like, are made of at least two parts which may be connected when the removable section is attached to the permanent section. However, in other embodiments of the full cross section removable sections, there may still be some materials or equipment which make their way only through the permanent section, which has one or more protruding parts going into and through the removable section.

In other embodiments, all cross sections of the removable section are substantially partial to the cross sections of the tip, such that at least one of the channels going through the tip are not split and are fully contained within the permanent section.

It will be appreciated that when the removable section is attached to the permanent section, all channels and flows which are split between the permanent section and the removable section, are securely connected such that no tool, material or energy may leak between the parts, and that all data may be continuously transferred.

In some embodiments, the removable section may be attached to the permanent section in a secure manner which will ensure that the removable section will not mistakenly disconnect from the permanent section within the body. A verification mechanism may be provided which adds extra security measures.

One technical effect of embodiments of the disclosed subject matter relates to providing an endoscope with removable tip section. This enables the medical stuff to replace the tip section of the endoscope in accordance with the required functionality, so as to use for each type of endoscopic session the most suitable endoscope configuration, equipment, size, or the like. Different removable sections may then be used according to varying needs, thus eliminating the need for purchasing and maintaining multiple endoscopes for different applications. Thus, different removable sections may be of different configurations, for example have the image capturing components, light sources, or working channels located at different locations on the removable section, thus adjusting to the specific body cavity explored or to possible findings within the body cavity. In other embodiments, the relative location between the image capturing components and the light sources may differ. In yet other embodiments, different removable sections may contain different types of cameras, differing for example in their wave length, lens assembly, sensor or other parts, pointing directions, field of view, or other parameters. The light sources may also differ between different configurations, in order to provide the type of light which the used sensor is sensitive to. Different removable sections can be made to adjust to different patients, for example removable sections can be manufactured in different sizes for adults, children or babies. Different removable sections can also be used when different view fields, different viewing angles or different optical characteristics are required, for example in some situations a viewing angle of 170° may be used, while in situations that require viewing more details of a smaller area a viewing angle of 140° can be used.

Another technical effect of relates to providing a disposable removable section, thus eliminating the need for sterilization or reprocessing and reducing contamination risks.

Yet another technical effect of the disclosed subject matter, according to some embodiments, relates to providing a removable section which can be made personalized in order to provide good results for a particular patient.

Yet another technical effect of the disclosed subject matter, according to some embodiments, relates to the replaceable top enabling a health care facility to maintain only a small number of endoscope systems, thus reducing cost and maintenance, while using the most appropriate endoscope for each type of endoscopic session, each patient, or the like.

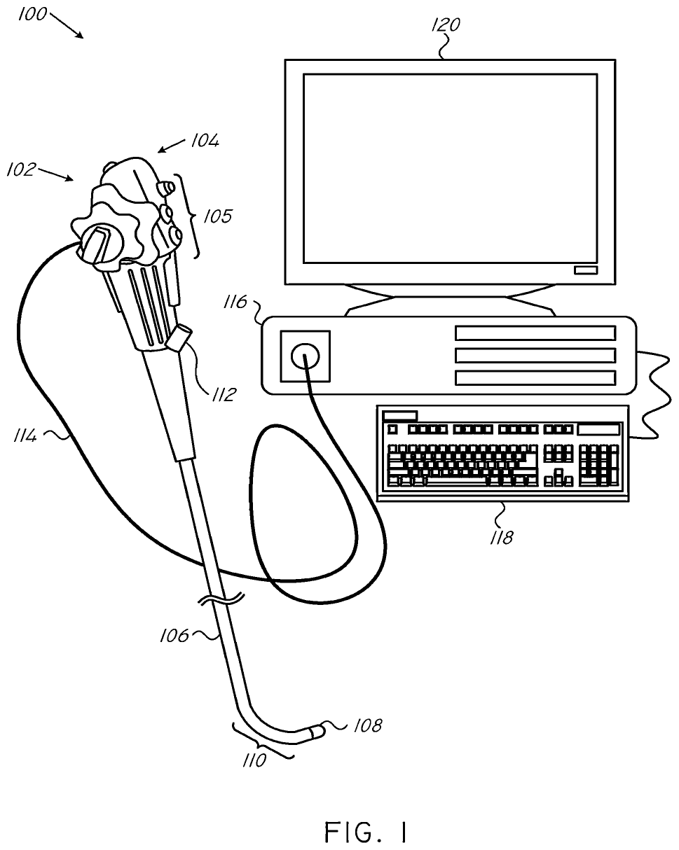

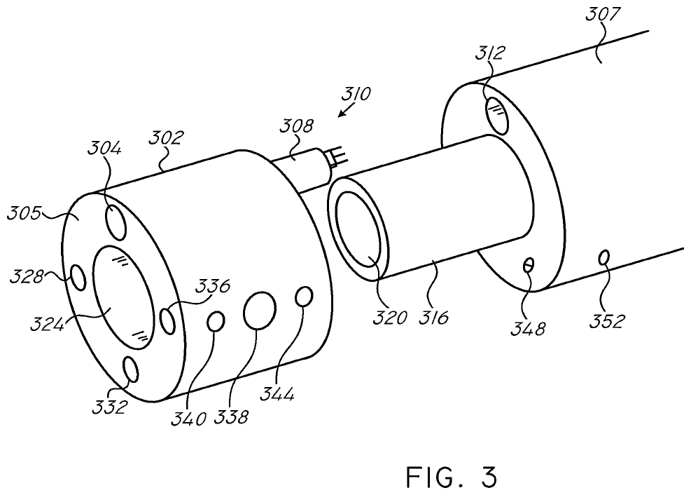

Referring now to FIG. 1, showing a semi-pictorial view of an endoscopy system 100. System 100 may include a removable tip endoscope 102, such as the removable tip endoscope of FIGS. 3 and/or 4. Removable tip endoscope 102 may include a handle 104, from which an elongated shaft 106 emerges. Elongated shaft 106 terminates with a tip section 108 which may be turnable by way of a bending section 110. Handle 104 may be used for maneuvering elongated shaft 106 within a body cavity; the handle may include one or more knobs and/or switches 105 which control bending section 110 as well as endoscopic functions such as fluid injection and suction. Handle 104 may further include one or more working channel openings such as working channel opening 112 through which surgical tools may be inserted.

A utility tube 114 may connect between handle 104 and a controller 116. Utility tube 114 may include therein one or more fluid channels and one or more electrical channels. The electrical channel(s) may include at least one data cable for receiving visual signals such as video signals from the camera or cameras on removable tip 102, as well as at least one power cable for providing electrical power to the cameras and to the discrete illuminators.

Controller 116 may govern power transmission to removable tip section 108 of endoscope 102, such as for the tip section's cameras and illuminators. Controller 116 may further control one or more fluid, liquid and/or suction pump which supply corresponding functionalities to endoscope 102. One or more input devices, such as a keyboard 118 may be connected to controller 116 for the purpose of human interaction with the controller. In another configuration (not shown), an input device, such as a keyboard, may be integrated with the controller in a same casing.

A display 120 may be connected to controller 116, and configured to display images and/or video streams received from the cameras of removable tip endoscope 102. Display 120 may further be operative to display a user interface for allowing a human operator to set various features of system 100.

Optionally, the image streams received from the one or more cameras of removable tip endoscope 102 may be displayed on display 120. If removable tip endoscope 102 comprises multiple cameras, the images may be displayed on display 120 side-by-side, interchangeably (namely, the operator may switch between views from the different cameras manually), or in any other manner. Alternatively, the video streams may be processed by controller 116 to combine them into a single video frame which may be panoramic or three-dimensional, based on an overlap between fields of view of the cameras or on known coordinates obtained for example from fiducials.

In another configuration (not shown), two or more displays may be connected to controller 116, each for displaying a video stream from a different camera of the multi-camera endoscope.

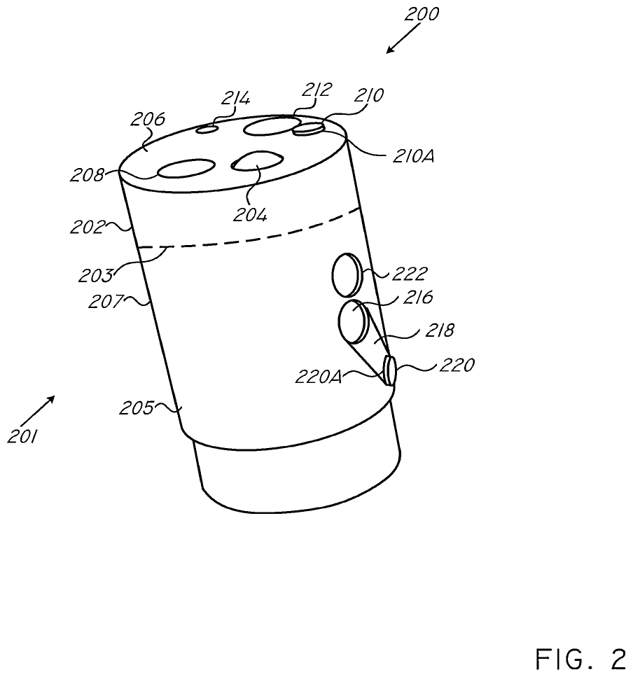

Referring now to FIG. 2, showing a perspective view of a removable tip endoscope.

Endoscope 200 may include an elongated shaft (not shown), a bending section (not shown) and a tip section 201 which terminates the endoscope. The bending section may enable the turning of tip section 201 in different directions. Tip section 201 may comprise a removable section 202 and a permanent section 207 connected along line 203.

Removable section 202 may include therein a front-pointing capturing device such as a camera or a video camera 204 which may capture images through a hole in a distal end surface 206 of tip section 201. A discrete front illuminator 208, which is optionally a light-emitting diode (LED), may be associated with front-pointing camera 204 and used for illuminating its field of view through another hole in distal end surface 206. The LED may be a white light LED, an infrared light LED, a near infrared light LED or an ultraviolet light LED. The light may be generated internally within endoscope tip section 201, or generated remotely and transferred, for example, by a fiber optic. In some embodiments, removable section 202 may comprise two or more illuminators, wherein at least one may generate the light internally, and at least one may provide remotely generated light.

A front fluid injector 210 may be used for cleaning at least one of front-pointing camera 204 and discrete front illuminator 208. Front fluid injector 210 may be slightly elevated from distal end surface 206, to enable it to inject fluid, from its side 210a, onto front-pointing camera 204 and discrete front illuminator 208. Front fluid injector 210 may be configured to inject fluids such as water, air and/or the like.

Distal end surface 206 may further include a hole defining a working channel 212. Working channel 212 may be a hollow tube configured for insertion of a surgical tool to operate on various tissues. For example, miniature forceps may be inserted through working channel 212 in order to remove a polyp or sample of which for biopsy. In alternative embodiments working channel 212 can be used for applying suction for evacuating various liquids and/or solids which exist in the body cavity and interfere with the inspection. In some embodiments, opening 212 can extend to an internal cylinder which comprises a part of permanent section 207.

A pathway fluid injector 214, defined by another hole in distal end surface 206, may be used for inflating and/or cleaning the body cavity into which endoscope 200 is inserted. Inflation may be performed by flowing air or another gas through pathway fluid injector 214, and may be beneficial for cases in which the body cavity, such as the colon, is shriveled or otherwise does not allow for efficient inspection. Cleaning may be achieved, for example, by injecting a liquid, such as water or saline, on an unclean area of the body cavity Furthermore, pathway fluid injector 214 (or a different tube, not shown) may be used for applying suction, in order to evacuate various liquids and/or solids which exist in the body cavity and interfere with the inspection.

Permanent section 207 of tip section 201 may include therein a side-pointing camera 216 which may capture images through a hole in a cylindrical surface 205 of the permanent section 207 of tip section 201. A side illuminator 222, which is optionally similar to front illuminator 208, may be associated with side-pointing camera 216 and used for illuminating its field of view through another hole in cylindrical surface 205. A side fluid injector 220 may be used for cleaning at least one of side-pointing camera 216 and discrete side illuminator 222. In order to prevent tissue damage when cylindrical surface 205 of permanent section 207 contacts a side wall of the body cavity, side fluid injector 220 and side-pointing camera 216 may be located in a notch 218 in the cylindrical surface. This way, side fluid injector 220 may be elevated from depression 218 but still not significantly protrude from the level of cylindrical surface 205. The elevation of side fluid injector 220 may enable it to inject fluid, from its opening 220a, onto side-pointing camera 216. In an alternative configuration (not shown), one or more discrete side illuminators may also be included in the depression, so that fluid injected from the side fluid injector may reach them. In yet another configuration (not shown), a side-pointing camera, one or more side illuminators and a side fluid injector may not be located in a depression, but rather be on essentially the same level as the cylindrical surface of the tip section.

It will be appreciated that the division of tip section 201 into removable section 202 and permanent section 207 shown in FIG. 2 is schematic only and is intended as a general demonstration. The cameras, working channels, illumination channels, fluid injectors and other components may be split between removable section 202 and permanent section 207 in any other manner as demonstrated in the exemplary embodiments detailed in association with FIG. 3 to FIG. 6 below.

It will be appreciated that further flexibility may be provided if any of the capture devices (such as cameras), working channels, illumination channels and other components are provided on the removable section rather than on the permanent section. In such arrangement, each removable section is configured and equipped with the camera types and other equipment and arrangement which are most appropriate for the task. However, some equipment such as cameras of higher quality and price may be located on the permanent section, so as to better utilize such resources in multiple application types.

Referring now to FIG. 3, showing a perspective view of a substantially full cross section of a removable tip, removed from the permanent section.

Removable section 302 of a tip of an endoscope is shown removed from permanent section 307, wherein permanent section 307 is connected to a shaft (not shown).

Removable section 302 may comprise one or more capture devices for example video camera 304, one or more light sources such as light source 328, or one or more fluid injector such as 332 or 336.

One or more cables providing power to camera 304 and transferring images from camera 304 to the shaft go through removable section 302, into and through an elongated section 308 protruding from removable section 302. When removable section 302 is connected to permanent section 307, elongated section 308 enters a corresponding recess 312 in permanent section 307. In some embodiments, elongated section 308 may end with a connector, wherein recess 312 contains a corresponding connector, such that when elongated section 308 is entered into recess 312 the two connectors connect such that power or data can flow between the endoscope and camera 304. For example, a plug located at the end of elongated section 308 may enter a corresponding socket inside recess 312. In alternative embodiments, recess 312 may comprise a plug and elongated section 308 may comprise a socket.

Thus, electric signals or data may pass through elongated section 308 and recess 312 from the shaft to the camera.

In some embodiments, elongated section 308 may protrude from permanent section 307 while recess 312 may be placed on removable section 302.

It will be appreciated that removable section 302 or permanent section 307 may comprise additional one or more pairs of protruding sections and corresponding channels, for transferring water or other fluids or liquids, optic fibers or any other material or equipment. When the protruding sections and corresponding channels are used for transferring fluids or liquids, one or two of them may be constructed with gaskets for sealing the fluids or liquids and avoiding leakage into the body or into other parts of the endoscope tip, from a gap between removable section 302 and permanent section 307.

Permanent section 307 may also comprise a hollow elongated section 316 protruding therefrom containing channel 320. When removable section 302 is connected to permanent section 307, hollow elongated section 316 is inserted into a corresponding channel 324 in removable section 202, which extends through the entire length of removable section 302, thus enabling a surgical tool (not shown) to pass through a working channel extending from the shaft through channel 320 of hollow elongated section 316 and through channel 324 in removable section 302 to distal surface 305 of removable section 302, so that the surgical tool can be used for operating on the body cavity of the patient.

Removable section 302 may also comprise one or more side-pointing capturing devices such as camera 338, one or more light sources 340 or one or more fluid injectors 344. The utilities to camera 338, light source 340 or injector 344, may be received from the same provisioning as the front facing camera, light sources and injectors, through corresponding pipes within the body of removable section 302 around channel 324. The images captured by camera 338 may also be transferred through the same channels.

It will be appreciated that removable section 302 or permanent section 307 may comprise additional side pointing cameras, light sources or injectors.

Removable section 302 and permanent section 307 may be connected by any known mechanism, such as locking mechanism, fastening mechanism, snap mechanism, or the like.

Removable section 302 or permanent section 307 may be equipped with a button 352 for releasing the connection. In order to avoid harming the body cavity of the user, button 352 may be placed within a recess so as not to protrude from the surface of the tip section. In some embodiments, the connection may only be released if a corresponding command is provided from an external source, such as simultaneous clicking on a control on display 120 which may be translated to an electrical or mechanical effect required for releasing the connection, in order to prevent unwanted accidental release.

In some embodiments, permanent section 307 may comprise a button or another sensitive area such as switch 348 which may be touched or pressed by removable section 302, only when removable section 302 is securely connected to permanent section 307. Such button may also be electrically connected to the endoscope handle or controller and may provide an indication to the endoscope operator whether the parts are securely connected. The indication may be visual, such as an icon on display 120. In some embodiments, when the connection is released, a vocal indication may also be provided as well to alert the operator.

In some embodiments, there may be two degrees or two mechanisms of connection between removable section 302 and permanent section 307. If one degree or one mechanism is released while the endoscope is being used, the operator may receive a first alert so he or she can remove the endoscope or otherwise correct the situation before the removable section is released within the body cavity of the patient.

It will be appreciated by a person skilled in the art that if the endoscope comprises an optic fiber, then each of removable section 302 and permanent section 307 may comprise a part of the fiber, wherein the sections may comprise corresponding lenses for providing continuity between the fiber parts by transferring light.

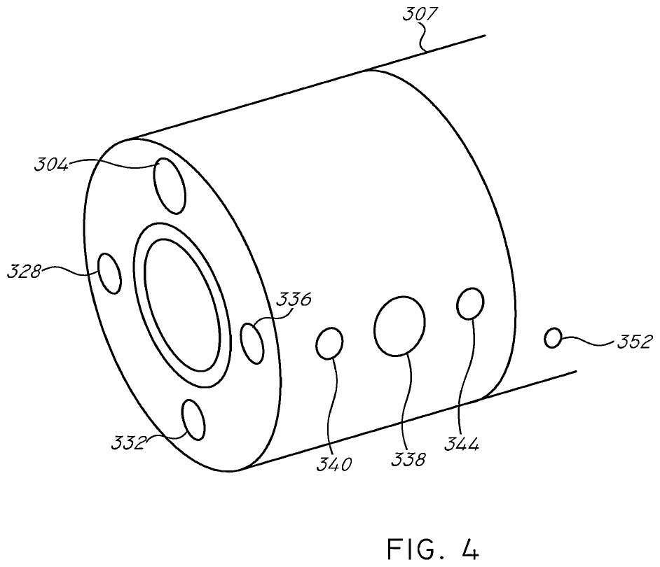

Referring now to FIG. 4 showing a perspective view of a substantially full cross section removable tip section attached to the permanent section.

In FIG. 4, removable section 302 is fully connected to permanent section 307, such that elongated section 308 and hollow elongated section 316 of FIG. 3 are inserted into corresponding recess 312 and channel 324, respectively. Electric signals or energy as well as water or fluids may pass through permanent section 307 to removable section 302, and images captured by the cameras are transferred back and may be displayed to an operator.

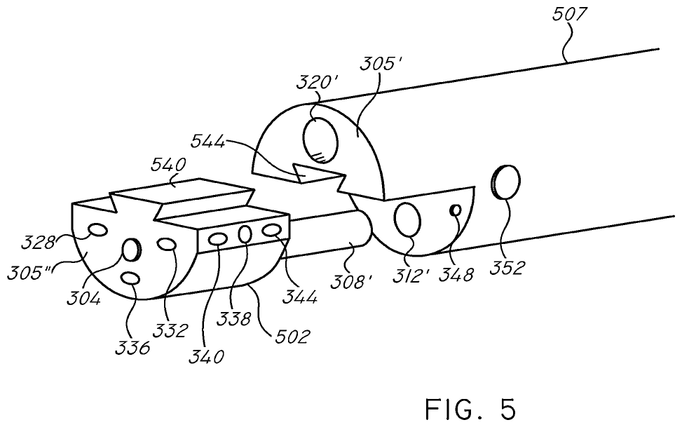

Referring now to FIG. 5 showing a perspective view of a partial cross section removable tip section.

In FIG. 5, distal face 305 of the endoscope tip is comprised of two parts, wherein a first part 305′ of distal face is of permanent section 507, while the other part 305″ is of removable section 502. Thus, each cross section of removable section 502 comprises a partial cross section of the tip section when assembled of the two sections. In the exemplary embodiment of FIG. 5, channel 320′ fully contained within permanent section 507 forms a working channel and reaches through permanent section 507 to the distal face so that tools or other equipment can be passed.

Removable section 502 may be equipped with cameras 304 or 338, light sources 328 or 340, or one or more fluid injector 332, 336 or 344 which may be located at the front face or on side face of removable section 502 as required.

The cameras, light sources or fluid injectors may be implemented and receive utilities as detailed in association with FIG. 3 above.

Removable section 502 may also comprise one or more elongated sections such as elongated section 308′ which fits into recess 312′ of permanent section 507. The one or more elongated sections such as elongated section 308′ may function as anchoring mechanism to secure removable section 502 within permanent section 507. Alternatively or additionally, the one or more elongated sections such as elongated section 308′ may be used for transferring electric energy, fluids, liquids, optic fibers or other equipment or materials between removable section 502 and/or surface 305″ and the endoscope handle and/or console.

In order to provide for full and tight connection between removable section 502 and permanent section 507, removable section 502 may comprise a trapeze shaped bulge which fits into recess 544 of permanent section 507. In alternative embodiments, removable section 502 may comprise a recess and permanent section 507 may comprise a bulge.

Permanent section 507 and removable section 502 may be connected in any required manner as detailed in association with FIG. 3 above.

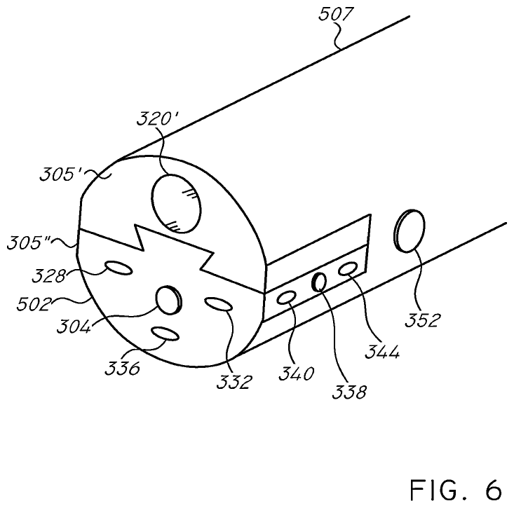

Referring now to FIG. 6 showing a perspective view of a partial cross section removable tip section attached to the permanent section.

When removable section 502 is securely attached to permanent section 507, first part 305′ of the tip section distal face which is part of removable section 502, and second part 305″ of the tip section distal face which is part of permanent section 507 are substantially on the same plane with minimal or no gap therebetween, and complement each other to create the full distal face of the tip section. When removable section 502 and permanent section 507 are securely attached, switch 348 of FIG. 5 may be pressed to provide an indication to an operator of the endoscope. Removable section 502 and permanent section 507 may be released by pressing button 352, with or without providing an external release command.

When removable section 502 is securely attached to permanent section 507, utilities and equipment may be passed through a working channel formed by channel 320′ and through elongated section 308′ and corresponding channels in permanent section 507.

It will be appreciated that the disclosure is not limited to a single removable sections, and various implementations can be designed in which two or more removable sections are used, for example to further increase the modularity and flexibility as appropriate for each application type.

While a number of exemplary embodiments and configurations have been discussed above, those of skill in the art will recognize certain modifications, permutations, additions and sub-combinations thereof. It is therefore intended that the following appended claims and claims hereafter introduced be interpreted to include all such modifications, permutations, additions and sub-combinations as are within their true spirit and scope.

In the description and claims of the application, each of the words “comprise” “include” and “have”, and forms thereof, are not necessarily limited to members in a list with which the words may be associated.

1-20. (canceled)

21. An endoscope system comprising a shaft defined by a longitudinal axis, wherein the shaft comprises:
a first section including a first distal portion, wherein the first distal portion includes a distally-facing front surface, wherein the distally-facing front surface includes a recess; and
a removable section connectable to, and removable from, the first section, wherein the removable section includes a connection section protruding proximally from a remainder of the removable section, and wherein the connection section is configured to enter the recess and provide an electrical connection between the first section and the removable section.

22. The endoscope system of claim 21, wherein the electrical connection is configured to pass electrical signals or data.

23. The endoscope system of claim 21, the first section comprises a first electrical connector in the recess, and the connection section comprises a second electrical connector that is configured to connect with the first electrical connector.

24. The endoscope system of claim 23, wherein the first electrical connector comprises a plug, and the second electrical connector comprises a socket.

25. The endoscope system of claim 21, wherein the removable section includes a distally-facing surface that includes an image sensor.

26. The endoscope system of claim 21, wherein a perimeter of the distally-facing front surface is flush with a perimeter of the removable section when the first section is connected to the removable section.

27. The endoscope system of claim 21, wherein the first section includes a channel section protruding from the distally-facing front surface, the channel section includes a channel, and a distal surface of the channel section is flush with a distal surface of the removable section when the first section is connected to the removable section.

28. An endoscope system comprising a shaft defined by a longitudinal axis, wherein the shaft comprises:
a first section including a connection section protruding distally from a distally-facing front surface of the first section, and
a removable section connectable to, and removable from, the first section, wherein the removable section includes a proximally-facing recess, wherein the connection section is configured to enter the proximally-facing recess and provide an electrical connection between the first section and the removable section.

29. The endoscope system of claim 28, wherein the electrical connection is configured to pass electrical signals or data.

30. The endoscope system of claim 28, the removable section comprises a first electrical connector in the proximally-facing recess, and the connection section comprises a second electrical connector that is configured to connect with the first electrical connector.

31. The endoscope system of claim 30, wherein the first electrical connector comprises a plug, and the second electrical connector comprises a socket.

32. The endoscope system of claim 28, wherein the first section includes a proximally-facing surface that includes an image sensor.

33. The endoscope system of claim 28, wherein a perimeter of the distally-facing front surface is flush with a perimeter of the removable section when the first section is connected to the removable section.

34. The endoscope system of claim 28, wherein the first section includes a channel section protruding from the distally-facing front surface, the channel section includes a channel, and a distal surface of the channel section is flush with a distal surface of the removable section when the first section is connected to the removable section.

35. An endoscope system comprising a shaft defined by a longitudinal axis, wherein the shaft comprises:
a first section including (i) a working channel extending to a distalmost end of the first section, and (ii) a recess defined by a distally-facing surface of the first section, wherein the distally-facing surface is proximal of the distalmost end of the first section, and
a removable section connectable to, and removable from the first section, wherein the removable section includes (i) a lumen extending longitudinally entirely through the removable section, (ii) a distalmost end including an opening of the lumen, and (iii) a connection section protruding proximally from a remainder of the removable section, wherein the connection section is configured to enter the recess and provide an electrical connection between the first section and the removable section, wherein the working channel extends entirely through the lumen, and the distalmost end of the removable section is flush with the distalmost end of the first section, when the removable section is coupled to the first section.

36. The endoscope system of claim 35, wherein the electrical connection is configured to pass electrical signals or data.

37. The endoscope system of claim 35, the first section comprises a first electrical connector in the recess, and the connection section comprises a second electrical connector that is configured to connect with the first electrical connector.

38. The endoscope system of claim 37, wherein the first electrical connector comprises a plug, and the second electrical connector comprises a socket.

39. The endoscope system of claim 35, wherein the removable section includes a distally-facing surface that includes an image sensor.

40. The endoscope system of claim 21, wherein a perimeter of the first section is flush with a perimeter of the removable section when the first section is connected to the removable section.
